SEQUENCE LISTING (9 sequences)

SEQ ID NO: 1 - protein, 73 aa, Homo sapien
MLGLVLALLSSSSAEEYVGLSANQCAVPAKDRVDCGYPHVTPKECNNRGCCFDSRIPGVPWCFKPLQEAECTF

SEQ ID NO: 2 - protein, 84 aa, Homo sapien
MATMENKVICALVLVSMLALGTLAEAQTETCTVAPRERQNCGFPGVTPSQCANKGCCFDDTVRGVPWCFYPNTIDVPPEEECEF

SEQ ID NO: 3 - protein, 106 aa, Homo sapien [sequence omitted]

SEQ ID NO: 4 - DNA, 222 bp, Homo sapien
atgctggggctggtcctggccttgctgtcctccagctctgctgaggagtacgtgggcctgtctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcagaatgcaccttctga

SEQ ID NO: 5 - DNA, 255 bp, Homo sapien
atggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttag

SEQ ID NO: 6 - DNA, 390 bp, Homo sapien
atgggacggcgagacgcccagctcctggcagcgctcctcgtcctggggctatgtgccctggcggggagtgagaaaccctccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaagactgccattactaa

SEQ ID NO: 7 - DNA, 3280 bp, Homo sapien
atgctggggctggtcctggccttgctgtcctccagctctgctgaggagtacgtgggcctgtgtgagtactgccctgactgccccggtggcagggtgggcgtgaagggaagggatccaggataaggggggattctgcattcatttaataatggccacctgtcacatatacactttttcctgcgctagccctttgaagtgggtctttattgtccccatttcacagacaaggaaaccgaggctcagagaaagttaacaacttatccaaggcagccctgcccagtctgtgttgaaatcagggtttgagcctgagcccatcccctatgaccccatagccatctttgctggagatttctaaattacaatataggtctttatgcattgttccacatttacaaagaaaaaggaaagatgcaggagaaaaaccctgacttcagaacactgtcaataccggcaggcacaaggttcatttagccattgcatagcaaccctgccatggggtgtggctgctccattaacccaagtttgaaggaatgagggcatggcttttatctgggtgtcttctgagcagggtcaaaggcagtggttcccgaacttgcagcccattagaatcacctggagagctttaaaaatcctaatgcttggggcacaccagttacatcagggcatctccaggcaagatccaggcctcagctgttttgttttgagatagccttgctttgtcactcactgctggagtgcagtggcacaatctcagctcactgcaacctccgcctcctgggttcaagcaattcttgtgcctcggcttcaagtagctgggattacaggcatgcaccaccatgcccagctaattttttggatttttagtagagatggagtttcgctatgttggccaagctggtctcaaactcctggcctcaagtgatcctcctgccttggcctcccaaagtgctggaattacaggtgtaagccaccatgcccagccaacgtcagtcatttttaaagctctgcagctgattccagtgtgagcgaagtttggatgccaggaggataagcaattacggactgggagcaagagaagggaatgtaagacactgcacgtgattgccattttcctaaggaaatactcagttcgttaatgaaacgcagtgaacttctgctgcacatacagacatagaggcttgcctgaaacatgaaaatattggggactgaaggatgtcccgggagggtgggacatgctcaacaattcaggaaggggagatgcagaaaaaagtgaaaagcaggcagcatgcgttgcaatgatctctatggcgtgtgcctctcctgtcacggttttcatttaaaacaaaggggcaaggttttgttggtcaaacaatgaagggtaactttgtttctgggttcaagggaccccagattccccaggggttcctgccagctggaaggtacccaggtccgtatgtgacttcccgagaaggtgataagagcgtgccaaggagaaagacacttaggcaaatggccagagtccccgagctgagcatttaacagactgcctctctttaaatattcacagggaaagtgcatcttcctaagggcgagggtttcagcagtggttgaactcggcggggtggggcggagcgggaggatgcaaacttgcaaagtgaagcaaacacactcaccgcagcccagcaagggctctggcagctgacagggctttgtctgggacagctgcaaaccagtgtgccgtgccagccaaggacagggtggactgcggctacccccatgtcacccccaaggagtgcaacaaccggggctgctgctttgactccaggatccctggagtgccttggtgtttcaagcccctgcaggaagcaggtaaggccccagtggcatcgtggtctgggcccagccccataaggcagggggtctcagggcctccctgtcctttctgggctggagatggaggcacaaggaccccaggaagccacacacacacacctgttccaaggcctcagagcagaggcttcacacttagggcagccatggccaggggctgtcctcttctgtcccctttatgtaaaacataaaagcaattgtttcaaaaaggtgttcaaaatgatggcatcgcatagagggaactgatttagtaactattcttgagagaagtggaaacgcataggtgtggaaagccgggccgacttttgggctgtttttgcaaatcggccccccagagtcttgtcatttgtggcatcccctacacagacggcaggcggtcccagccctagacgtcaggcctcggtgccacaccccacctcccccactctgccccccacaagggtcatctcctctccctctctctgccgtggtggagggcaggtgcagggcaaccaccctgggggttccctccccaggggcggagagcctgcgtgctgtgcgggtaacagatggccctgcacacgggtttgccaccctggctccaccaggcttagctgccccacatcgtgggtggggcgattggctataagccatctgccatgtccaagtgccagctcagcccccacgaaggccgcacctgcgtgaggtaccttcctggaaccagcatccagaggggcctctcttgccctttgtcctagggtgaaatgcgggaggctgagtcctgctggccccggctccctgatcaatgatgggcccctgcccagggcctcccttcaccctccccagcaagtccagggtaggggtgggggtgggggtccagagaaggccaggagagagaggggtctggctactgtccactgccggtcctgttccttcagctccactggaactacactctcctctgagtgccagccatggccctgccaaggcccatctcgcttgttatctgcctgatccctgggtcccactatcttgcttagcaacccgaggtgggaatcttggctattcccccatgtggtggggactcaacactccccggtgactctggggaggaggcagcactaggtgctggccttggagcctgccctgaccttgggaagctgggcagcgtgggtggagagagactgctcacacaagcctttgctctgtttgcagaatgcaccttctgaggcacctccagctgcccccggccgggggatgcgaggctcggagcacccttgcccggctgtgattgctgccaggcactgttcatctcagcttttctgtccctttgctcccggcaagcgcttctgctgaaagttcatatctggagcctgatgtcttaacgaataaaggtcccatgctccacccg

SEQ ID NO: 8 - DNA, 4623 bp, Homo sapien
dccctggggtgcagctgagctagacatgggacggcgagacgcccagctcctggcagcgctcctcgtcctggggctatgtgccctggcggggagtgagaaaccctgtaagtgaaggagagggtctttttatgtgctttctttatttctcttaaagaaaaaaaaaaagcacaaccataaattaacttgagagggggaatggctataaaggcatctggcaatgtgtgttgttcacatgggatttgccactgctcaggagggtggctccaagaagggcctccctcctagggaaaggctgagtgacggcaggtgtcagcgggccccgtgtcgggccaggagggcattcccaccaagggtccttggagtcccagagcactcacctctcgcctggatcttggccttgggtccatctgttcaccctcctctaggagggttttgtttttgtttttttccgagacaggatctggctttgccgcccaggcaggagtgcagtggtgtgatcttggctcactgcaacctctgcctcccaggctcaagtgatcctcccacctcagccgcctgagtagctgaaaccacagttgtggaccatcatgcccggccaatttttttttttgtattgtttgtagagatggggtttcgacatgttgcccaggatggtcttgaactcctgagctcaagcaatctgcccgcctcggcttcctaaagtgctgggattataggtatgagccaccatgcctggcttttttttttttttccttttaaactaatataacaatttcagcaaagccctatcggcttctcaggaggaaaccgcattgcttaaatatgggcaagataagactttgtgtttctctatgtggcaacaagacagtagaggcatcccctagaacctctgagagaaggagcagtgtggtctggggtaccagggtggggccgactgagggtctttccacagccccctgccagtgctccaggctgagcccccataacaggacgaactgcggcttccctggaatcaccagtgaccagtgttttgacaatggatgctgtttcgactccagtgtcactggggtcccctggtgtttccaccccctcccaaagcaaggtaatcttccagggaatcttcctgggccagcagctggcaacccaggacccagcttcacaggcggagcccagagcaggggccggaggaggcccagttgctagtctagggttagcctgggtgggttagtctcgagctagccccggttggttagtctggggctagcccaggttggttagtctagagctagcccaggttggttagtctggggctagcccaggttggttagtctggggctagcccaggttggttagtctagggctagtgtaggctagttagtctaaggctagcccaggttggttagtttggagctagcgcaggttggttagtctggggctagtagcccaggttggttagcctggagctagcccaggttggttagtctagggctagcgtaggctggttagtctggggctagcccaggttggttagtctggagctagcccaggttggttagtctggggctagtagcccaggttggttagtctggggctagcccaggttggttagtctagggctagtgtaggctagttagtctagggctagcccaggttagttagtttggagctagcacaggttgattagtctggggctagtagcctaggttggttagtctggagctagcccaagttggttagtctagggctagcataggctggttagtctggggctagtagcctaggtttgttagtctggagctagcccaggttggttagtctagggctagcgtaggctggttagtctagggctagcccaggttggttaatcggagctagcccaggttggttagtctggagctagcccaggttggttagtctgaggctagtagcccaggttggttagtctggggctagcccaggtttgttagtctggagctagcccaggttgtttagtctggagctagcccaggttggttagtctgggactagcctggactgctagtctagaggtagcctagaggactgctagtctagaggtagtctagggctagcccaggttggttagtctggggctagcccatgttggttagtcttagactagcctggactgctagtctagaggtagcccaggttgtttagtctggtactagcctggactgttagtctagaggtagcccaggttggttaggttggttagtctgggactagtctggactgttagtctagaggtagcccaggttggttagtctgggactagcctggactgttagtctagaggtagcccagattggttagtctgggactagtctggactgctagtctagaggtagcccaggttggttagcctggggccagcctggactgttagtctagaggtaacccaggtcagccaacagtgagatgaaaatttcccacctaccctgtttctacactgttagttctttcaacagacatgtgtgtgtggagccatcagttttactttagttgagaaaaaaatatatatatatatagtaggtctcctctagtttttgaagtgtgacttctgaagaagcttccatggggaaatgaaggtatttaataggacagcagtaacataagggctgacagccctcaaatgttagggaaggaagtgaagccttctagggttctttgggagtgagttttatgttagtgcacgggatcaggacccaagttgtaacgccgacgagtgctcaaaggaaggttgtgtgtgtgtcgtgcacctgtgtgcgtggaaccaggcacgtcctctggagaaggaggattcatccccaagattgttgctgggaggcttgctgggccccgcagggaaaccaggcagatggtggattgttcacgagcgcccactgaatggcagtgtctttgggaatcaataccatgtccaaacgctttccatcttaccaaggtgcccacaaaccttttctcatcttggcccgggggaccaccccatttactgagaacactgagtcccgagaggcaaaatgatttccccaaggcgggggactccagagcttctgactgtgaccaccccacatgggccccaccttcgcggaggacaggccagccaagcgtcgctggggccgacacttccacagtccccgggggaggcggtcccaggggccgacacttccacagtccccgggggaggccgtcccgggggatgctgccccaggcagcacctcatgatccacggaggctgcaaatcagcgctgctctcagaggaggaaggggtggagctttccagggcacagcaggcctgactgggtctcggtgctgtgcctgtcccatggcagagtcggatcagtgcgtcatggaggtctcagaccgaagaaactgtggctacccgggcatcagccccgaggaatgcgcctctcggaagtgctgcttctccaacttcatctttgaagtgccctggtgcttcttcccgaagtctgtggaaggtaacgtcgctgtgggactctctgtctggttcccggacaccatgattcctcctccgtccgtagaggtggggtgcagggaggggagctgcctcgcagcctcagtgccatcgaggccagggcccctgcctcctatgggattctgaaggcaattccagaatgttcttggcaaagacagcgtcttttcaataagtttatagcctccagcattgccactgcgtcatctgtgatggctctagaaacagcggctcatccctgttgcctccccaggtgttgcaacgttcagaggcgttgcctgttttattgcaagcccatctgcatttggaggctactgagtgtcttgcactgtgctgggtaccagagagggcccaactcaagcagacctggccccttctcccgtggcttccccgttctcccccacatgaccccgaatgacaaacctcatccacaacgtcctgctccgggcagtcccgggagggtcccgccggcagaggtgaacgggtccacttctcccacccgcttagtgatagtgtgttcctgactcggagtgtggcgaggtaaaaaaagaccaagcagatccaggaaaatggggaaagagctactggcccttgaaggatgccttttcttttccttttgttaggatatcaaagcactccaaagagcgaaatatttcatgttcaggattttccgagtgattttttttatgtgacctaaaggtccacctagaaaatgttcacttgtctggggagaatgcgccccacagaggaaactctggcctggggtgggaagatttggtccctttacaccccctccccgggaaaggagctccttcttcagtaggaagctcctgggcaaagtgatgcacgcccaccccagcttcgcagcctaggcactcccatttctggggttcccttaccaaccatcttgcatttaaacttctagactgccattactaagagaggctggttccagaggatgcatctggctcaccgggtgttccgaaaccaaagaagaaacttcgccttatcagcttcatacttcatgaaatcctgggttttcttaaccatcttttcctcattttcaatggtttaacatataatttctttaaataaaacccttaaaatctgct

SEQ ID NO: 9 - DNA, 4252 bp, Homo sapien
atccctgactcggggtcgcctttggagcagagaggaggcaatggccaccatggagaacaaggtgatctgcgccctggtcctggtgtccatgctggccctcggcaccctggccgaggcccagacaggtaaggcgtgcttcttcctgctctgtggggccacagccagctctggcagcctccgccaggagccactgttttacatacatatttttgagcacctgttttgtgccaggtgctgttctaggcccttaaaagtatatccaatttacaggatcggcaaaagcaggtggagagtaactcagggtggcagggcccccggagaccttcgagaagtgcgacgaggagggggctgccttcagtcggggctgttttcctgtgttaggaagactatacaatcctcccaagtgtcatgtttcaaagaggaagtgttggcgtggggtctcagaatagtgcttttgactgttcatgccaacatctcccccaggggcagaccctcccaaggcccatccagataggcccaaatgccggtcccagtgatggccacctgggagaccctctcccacaggcccgaatgcccgtcccagtggtggccaactgggagaccctctcctacaggttcctgggctcccctgggatccatgctctgggagtcaaagccacctctctcatgagtgcgtggctggcaacccatattccctggtgttgtcaagtggatcggttgccctgggtccttctagggagtggaggaggaggccattcttgcttccttgggaagtgtttgcatctcaactcctttacctgcagaatggatcaacggtctgccctagggctgtcaggaaatgctgtgtggcagcatctgcgacttgcactttgccagctgtggggagctgaataacttatttgccgttattaggtacagtttcaaggtgggggcaggagaaagggctttctacgtttccaaagcaagggtttccagagaggcctgaagagggagcgcccagtggtgctgtccgtgcccccactgccctccagccacctcttgatctctgctgtggggtaccgggcctgaggggtgggcttgggcagcgtagaagagcagccagcattgggctgcagtgggaagacccccaagcccatggcagggagcgggggagctttggaacccgagagaggaagtggcctcggtgtacagaacgaactgggtgggtccccgtgctggccacccccaggcccatctgcctgcgcccttgcccccaccccagcccccagctctgccccctgtgctgtgggatcacagaggccgtggcaaactcccctccccaccccacacaccctctggctcaaggctcagagcgtctttgcgggtcactcaggtccatgatcctgttacaactgaaatctagaaaattgtgattacagtttagtgcattcgtgtgtggaaaccatttccatttatttccatcatgcgacaaagacaaagcgggtgggcaagacagagtctgccggaggcagagcaccggggctggaaatcttcctccctgaggaggaaacccccccgacccccaggatgatgatcctccctcaccacggggcctctcttgacccccacagtgtcccgggggtgggcgatgatcaccttcacgtcgcgatggatccagaccccaggagggcaaggttcccatggaagctgctgggcagcgggagctgaacacggatccttcccagcaagccaggaacactttctccaaagacatctcgaggcagtccctgatagcaaagcagacaagagaacagcccctctcggcctcccctggggcgccctcacctgagccagtgtggccagactgagttcctcccctcctatgccccaaggcagggacagggaccggagggtgctctgggctcctctttcaccccctgctgcaggctgtcaaccaccagatcctaataggttgctttctgagacctttgattccgcggagctcagagcctgaagctctggtgttagaacctcttgcataagatcctgcggcagcccccagccagccccatctgtccacgtgtcttcctcctctagatccctttcctcactgccctgcttcaagctgtttcacagcttgtaccctctgtcggctcctcctagaccaccccacccggtcctctcaccttacctgcaatgggtttccacctcctgaacacacctgggtctctggaatggcctttgcccatgcggctccatcttcacctggtgaacctcctcctgcagggagcccccctgctttgttcaacctgcttgtcattggcctctccggggagtgccctacccccgtggttaccctgggcaccctgggacgatggccttgcgttgtctcgcacatgttcttgcctttctcctccatcagatccttagactcttttttttttttttttgagatggagtcttgctctgtcactcaggctggagtgcaatggtgcgatcttggctcactacaacctctgcctcctgggttcaagtgattctcctgcctcagcctcccaagtagctgggattacagacgtgtgccacaatgcccgcctaattttttgtatttttagtagagatggggcttcaccattttggtcaggctggtcttgaactcctgacctcaagtgattcacctccttcagcctcccaaagtgctgggattacaggcatgagcctgggcccagatatttagactcttattaatgacttctctggttttaatttctgggtctctctcacctggcacagtgcctggcttttgccatgctagctcccacttctcatgcacacaaatggtgctcagtaaatatttatgtattgagtaaaatttaataatcatttgttgaaattaaaaagtgaataaataagttacctagaaagatgcaaagtccacaaacctggggcaccttgcattttccctgagcgtaatgtttgcacatcaggatgtgaggaccacgtctccctctcatgtcctgagggttttatatccgcctcactggacagttgctgatgtcattggagaaggaagctggatgggtgtgtgcatgataacatcaaggaattcagcccacaacttactttgcttcttacctgtgcactttcagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaaggtatggcctttttatacgatgggttctgaagatttagaattagttagaaaagtcatttaagactacagaggctctgatcagcatcaccagctatgcctttacacagagtcacggccgccagtggtggtgcaatggggtagcctgagtcaggctgcattcaggtccaggaatagaaaggcagggctaagggacttgggaagaaacctgatttccccccggcttctcttcacatctctaaccaaaagcctgggaagagccactgttggtaacgctttctagcttgcctaggatagagggggaaggcatgacgaaatctgaagacatttcatgtattctttttttttttttttttttgaaatggagtctcgctccgttgcccctgagctggagtgcaatggtgcgatcttggctcactgcaatctctgcctcctgagttcaacctcagcttcctagtagctgagattacaggtgtgtgccactacgcccagctaaattttttttgtatttttagtatagacggggtttcaccatgttggccagaccggtcttgaactcttgacctcaggtgatctgcccgcctcagcctcccagagagctgggattacaggcgtgagccaccgtgcccggctgacagttcatgttttctaaagaatgtgcctatggatactttaaagtaaaaactctgtaattgtttaaatgtgaaagaaaatgtttatcctcactaaagcatctctttctccctccccctcacccctgtagaggagtgtgaattttagacacttctgcagggatctgcctgcatcctgacgcggtgccgtccccagcacggtgattagtcccagagctcggctgccacctccaccggacacctcagacacgcttctgcagctgtgcctcggctcacaacacagattgactgctctgactttgactactcaaaattggcctaaaaattaaaagagatcgatatt

(19) United States
(12) Patent Application Publication
(10) Pub. No.: US 2008/0167230 A1
(43) Pub. Date: Jul. 10, 2008

(54) Methods and compositions for treating or preventing lesions of the respiratory epithelium

(75) Inventor: Daniel K. Podolsky, Wellesley, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 11/821,367

(22) Filed: Jun. 21, 2007

Related U.S. Application Data
(63) Continuation of application No. 10/305,747, filed on Nov. 27, 2002, now abandoned.
(60) Provisional application No. 60/333,836, filed on Nov. 28, 2001.

(51) Int. Cl. A61K 38/16; A61P 11/06

(52) U.S. Cl. 514/12

(57) ABSTRACT

This invention features methods of treating lesions of the airway epithelium by local or systemic administration of intestinal trefoil peptides. The intestinal trefoil peptide can be administered either alone or in combination with one or more therapeutic agents.

### CROSS REFERENCE TO RELATED APPLICATIONS

This application is a continuation application of 10/305,747, filed Nov. 27, 2002, which claims benefit of the filing date of U.S. Provisional Application No. 60/333,836, filed on Nov. 28, 2001. Each of the foregoing references is hereby incorporated by reference.

### FIELD OF INVENTION

This invention relates to methods and compositions for treating lesions of the airway epithelium that can result, for example, from viral, bacterial, and fungal infections, inflammation, allergens, inhaled organic solvents, particulates, or irritant gases.

### BACKGROUND OF THE INVENTION

Upper airway lesions, including lesions from the external nasal nares to the larynx, are caused by a wide variety of local irritants, allergens, and infectious agents. Typically, these irritants give rise to the symptoms of rhinitis or ‘runny nose.’ In cases of severe lesions however, the tight junctions of the respiratory epithelial mucosa are disrupted such that entry of allergens or infectious agents is facilitated.

Tracheo-bronchial lesions (trachea and conducting bronchial tubes to the level of the respiratory bronchioles) are also commonly caused by respiratory infections, irritants, and allergens. Once the tracheo-bronchial epithelium and tight junctions have been disrupted, infectious, irritant, or allergic material may sensitize the lung, triggering the release of mediators, and subsequent airway constriction and asthma.

The alveolar epithelium, distal to the respiratory bronchioles, is generally well protected against infectious, irritant, and allergic exposure. However, infectious, immunologic, or chemical agents that penetrate the deep lung structures can cause pneumonias. Infectious agents that gain access to the systemic circulation in the lower airway can further result in sepsis pneumonias or a respiratory distress syndrome. Moreover, in certain inflammatory conditions such as asthma, mucosal disruption results in increased levels of allergens and irritants, such that both inflammation and mucosal lysis are further exacerbated.

Rapid restoration of the normal airway epithelial barrier is therefore critical to reduce the damage caused by ongoing pathogenic or allergenic mechanisms in respiratory tissues and alleviate the associated symptoms.

### SUMMARY OF THE INVENTION

The present invention features methods and compositions for the treatment of lesions of the airway epithelium in mammals, by administering to the mammal therapeutically effective amounts of trefoil peptides, or a biologically active fragments thereof. Treatment of lesions according to the invention can speed healing, reduce pain, delay or prevent the occurrence of the lesion, and inhibit expansion, secondary infection, or other complications of the lesion. Lesions of the airway epithelium may result from any cause, including for example, an allergic reaction, asthma, an infection, an inhaled chemical or particulate exposure, a thermal lesion, smoke inhalation, drug-induced lung damage, trauma (caused, for example, by surgery or intubation), a microbial infection (e.g., bacterial, viral, or fungal), chronic obstructive pulmonary disease, anti-neoplastic therapy, cystic fibrosis, cardiovascular compromise such as congestive heart failure, or hyperbaric oxygen therapy.

In another aspect, the invention provides a composition, which includes a trefoil peptide in a pharmaceutically acceptable carrier suitable for inhalation administration. When formulated as such, the composition may be an aerosol (e.g., nasal spray, inhalation spray, inhalation solution, inhalation suspension) administered by a metered dose inhaler. If desired, the formulation containing the trefoil peptide may be nebulized (e.g., by jet, ultrasonic nebulizer, or electronic nebulizer). Alternatively, the trefoil peptide formulation may be administered as a dry powder using a metered dose inhaler or a dry powder inhaler, for example.

In all foregoing aspects of the invention, the mammal is preferably a human and the trefoil peptide is human intestinal trefoil factor (ITF), spasmolytic peptide (SP), pS2, or biologically active fragments thereof. Such fragments include for example, ITF₁₅₋₇₃, ITF₂₁₋₇₃, ITF₁₋₇₂, ITF₁₅₋₇₂, or ITF₂₁₋₇₂ of SEQ ID NO.: 1

In the methods and compositions of this invention, a second therapeutic agent can be included. Such agents include anti-inflammatory agents such as glucocorticoids (beclomethasone, flunisolide, budenoside, triamcinolone, prednisolone, dexamethasone, or fluticasone) or non-steroidal anti-inflammatory agents (e.g., ibuprofen, tacrolimus, cromolyn, nedocromil, refecoxib, or celecoxib); antimicrobial agents (e.g., amikacin, gentamicin, kanamycin, neomycin, netilmicin, paromomycin, streptomycin, or tobramycin); antihistamines (e.g., diphenhydramine, fexofenadine, cetirizine, or loratadine); cholinergic receptor antagonists (e.g., ipratropium bromide or tiotropium); neurokinin receptor antagonists; leukotriene receptor antagonists; decongestants; phosphodiesterase inhibitors; or beta-adrenergic receptor antagonists (albuterol, bitolterol, epinephrine, fenoterol, formoterol, isoetharine, isoproterenol, metaproterenol, pirbuterol, procaterol, racepinephrine, salmeterol, or terbutaline). The second therapeutic agent may be administered within (either before or after) 14 days, 7 days, 1 day, 12 hours, 1 hour, or simultaneously with the trefoil peptide.

The second therapeutic agent can be present in the same or different pharmaceutical composition as the trefoil peptide. When the second therapeutic agent is present in a different pharmaceutical composition, different routes of administration may be used. For example, the second therapeutic agent may be administered orally, or by intravenous, intramuscular, or subcutaneous injection. Thus, the second therapeutic agent need not be administered by inhalation.

Of course, pharmaceutical compositions may contain two, three, or more trefoil peptides or biologically active trefoil peptide fragments. Alternatively, inhalation of the trefoil peptide may be supplemented by systemic (e.g., oral or injectable) administration of the same or different trefoil peptide.

Airway epithelial lesions are prevented or ameliorated by administering the intestinal trefoil peptide-containing composition prior to the anticipated insult (e.g., surgery, or antineoplastic therapy for example). Preferably, the prophylactic treatment begins at least one day, three days, five days, seven days, or ten days prior to the insult. Treatment of unanticipated airway lesions preferably begin immediately after insult, or within 24 hours.

In a preferred embodiment, the trefoil peptide or biologically active fragment is encoded by an isolated nucleic acid sequence that hybridizes under high stringency conditions to a polynucleotide sequence having the sequence of SEQ ID NO: 4, SEQ ID NO: 5, SEQ ID NO: 6, SEQ ID NO: 7, SEQ ID NO: 8, or SEQ ID NO: 9.

In another aspect, the invention features a pharmaceutical composition suitable for inhalation administration, containing a trefoil peptide or biologically active fragment that is encoded by an isolated nucleic acid sequence that hybridizes under high stringency conditions to a polynucleotide sequence having the sequence of SEQ ID NO: 4, SEQ ID NO: 5, SEQ ID NO: 6, SEQ ID NO: 7, SEQ ID NO: 8, or SEQ ID NO: 9

Mammalian trefoil peptides were discovered in 1982. One of the mammalian trefoil peptides, human intestinal trefoil factor (ITF), has been characterized extensively, and is described in U.S. Pat. Nos. 6,063,755, and 6,221,840, hereby incorporated by reference. The other two known human trefoil peptides are spasmolytic polypeptide (SP) and pS2. Trefoil peptides, described extensively in the literature (e.g., Sands et al., Annu. Rev. Physiol. 58: 253-273 (1996), hereby incorporated by reference), are expressed in the gastrointestinal tract and have a three-loop structure formed by intrachain disulfide bonds between conserved cysteine residues. These peptides protect the intestinal tract from injury and can be used to treat intestinal tract disorders, such as peptic ulcers and inflammatory bowel disease. Homologs of these human peptides have been found in a number of non-human animal species. All members of this protein family, both human and non-human, are referred to herein as trefoil peptides. Human ITF will be referred to most extensively in this application; however, the activity of human ITF is common to each of the mammalian trefoil peptides.

The term “trefoil peptide” is meant to include all mammalian homologs of human spasmolytic polypeptide, human pS2 and human ITF polypeptides, and biologically active fragments thereof. Homologs of the trefoil peptides have, preferably, 70% amino acid identity to the human sequence, more preferably 85% identity, most preferably 95%, or even 99% sequence identity. The length of comparison sequences will generally be at least about 8 amino acid residues, usually at least 20 amino acid residues, more usually at least 24 amino acid residues, typically at east 28 amino acid residues, and preferably more than 35 amino acid residues.

The term “fragment” is meant to include polypeptides that are truncations or deletions of SP, pS2 and ITF. Preferably, the fragments have 70% amino acid identity to the corresponding regions of the human polypeptide sequence. More preferably, the fragments are 85% identical, most preferably 95%, or even 99% identical to the human polypeptide sequence to which they correspond. The length of comparison sequences will generally be at least about 8 amino acid residues, usually at least 20 amino acid residues, more usually at least 24 amino acid residues, typically at east 28 amino acid residues, and preferably more than 35 amino acid residues.

Preferable fragments contain four cysteine residues in any positions which correspond to the cysteines at positions 25, 35, 45, 50, 51, 62, or 71, of human ITF (FIG. 1), or positions 31, 41, 51, 56, 57, 68, and 82 of human pS2 (FIG. 2). More preferably, fragments contain five cysteine residues at these positions. Most preferably, six, or even all seven cysteines are present.

Fragments of SP are meant to include truncations or deletions and preferably have 70% sequence identity to the corresponding human SP polypeptide sequence (FIG. 3). More preferably, the fragments are 85% identical, most preferably 95%, or even 99% identical to the human polypeptide sequence. Preferably, active fragments contain at least four cysteine residues, which correspond to positions 6, 8, 19, 29, 34, 35, 46, 58, 68, 78, 83, 84, 95, and 104 in the human SP polypeptide. More preferably, fragments contain six cysteines, which correspond to these positions. Even more preferable are fragments that contain eight cysteines. Most preferable are fragments that contain cysteines at ten, twelve, or even, all fourteen positions.

It is recognized in the art that one function of the identified cysteine residues is to impart the characteristic three-loop (trefoil) structure. Accordingly, preferred fragments of ITF and pS2 have a least one loop structure, more preferably, the fragments have two loop structures, and most preferably, they have three loop structures. It is equally well recognized that the native SP polypeptide has a six loop confirmation. Preferable fragments contain at least two of these loop structures, more preferably, four loop structures are conserved, and most preferably, five, or even all six loop structures are present.

By “aerosol” is meant any composition of the trefoil peptide of the invention administered as an aerosolized formulation, including for example an inhalation spray, inhalation solution, inhalation suspension, a nebulized solution, or nasal spray.

By “antimicrobial agent” is meant any compound that alters the growth of bacteria or fungi cells, or viruses whereby growth is prevented, stabilized, or inhibited, or wherein the microbes are killed. In other words, the antimicrobial agents can be microbiocidal or microbiostatic.

By “antineoplastic therapy” is meant any treatment regimen used to treat cancer. Typical antineoplastic therapies include chemotherapy and radiation therapy.

By “biologically active,” when referring to a trefoil peptide, fragment, or homolog is meant any polypeptide that exhibits an activity common to its related, naturally occurring family member, and that the activity is common to the family of naturally occurring trefoil peptides. An example of a biological activity common to the family of trefoil peptides is the ability to restitute the gastrointestinal mucosa (Taupin et al., Proc. Natl. Acad. Sci. USA. 97(2): 799-804).

The term “isolated DNA” is meant DNA that is free of the genes which, in the naturally-occurring genome of the organism from which the given DNA is derived, flank the DNA. Thus, the term “isolated DNA” encompasses, for example, cDNA, cloned genomic DNA, and synthetic DNA.

The term “pharmaceutical composition” is meant any composition, which contains at least one therapeutically or biologically active agent and is suitable for administration to the patient. Pharmaceutical compositions suitable for delivering a therapeutic to the respiratory airways include, but are not limited to, aerosols and dry powders. Any of these formulations can be prepared by well-known and accepted methods of the art. See, for example, Remington: The Science and Practice of Pharmacy, 20^{th} edition, (ed. AR Gennaro), Mack Publishing Co., Easton, Pa., 2000.

By “high stringency conditions” is meant any set of conditions that are characterized by high temperature and low ionic strength and allow hybridization comparable with those resulting from the use of a DNA probe of at least 40 nucleotides in length, in a buffer containing 0.5 M NaHPO4, pH 7.2, 7% SDS, 1 mM EDTA, and 1% BSA (Fraction V), at a temperature of 65 C, or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well known by those skilled in the art of molecular biology. See, e.g., F. Ausubel et al., Current Protocols in Molecular Biology, John Wiley & Sons, New York, N.Y., 1998, hereby incorporated by reference. Other features and advantages of the invention will be apparent from the following detailed description, and from the claims.

By “substantially identical” is meant a polypeptide or nucleic acid exhibiting at least 75%, but preferably 85%, more preferably 90%, most preferably 95%, or 99% identity to a reference amino acid or nucleic acid sequence. For polypeptides, the length of comparison sequences will generally be at least 20 amino acids, preferably at least 30 amino acids, more preferably at least 40 amino acids, and most preferably 50 amino acids. For nucleic acids, the length of comparison sequences will generally be at least 60 nucleotides, preferably at least 90 nucleotides, and more preferably at least 120 nucleotides.

By “therapeutically effective amount” is meant an amount sufficient to provide medical benefit. When administering trefoil peptides to a human patient according to the methods described herein, an effective amount will vary with the size of the lesion area being treated; however, a therapeutically effective amount is usually about 1-2500 mg of trefoil peptide per dose. Dosing is typically performed one to four times each day. The patient may also be administered with a trefoil peptide continuously over a set period of time.

### BRIEF DESCRIPTION OF THE DRAWINGS

FIG. 1 is an amino acid sequence of a human intestinal trefoil factor (ITF; Accession No. BAA95531) (SEQ ID NO.:1).

FIG. 2 is an amino acid sequence of a human pS2 protein (Accession No. NP_{—}003216) (SEQ ID NO.:2).

FIG. 3 is an amino acid sequence of human spasmolytic polypeptide (SP; Accession No. 1909187A) (SEQ ID NO.:3).

FIG. 4 is a cDNA sequence encoding a human intestinal trefoil factor (SEQ ID NO.:4).

FIG. 5 is a cDNA sequence encoding a human pS2 protein (SEQ ID NO.:5).

FIG. 6 is a cDNA sequence encoding a human spasmolytic polypeptide (SEQ ID NO.:6).

FIG. 7 is the nucleotide sequence of a gene encoding human intestinal trefoil factor (locus 10280533:52117-55412) (SEQ ID NO.:7).

FIG. 8 is the nucleotide sequence of a gene encoding human pS2 protein (locus 10280533:16511-21132) (SEQ ID NO.:8).

FIG. 9 is the nucleotide sequence of a gene encoding human spasmolytic polypeptide (locus 10280533:957-5208) (SEQ ID NO.:9).

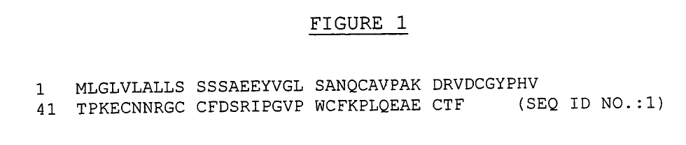

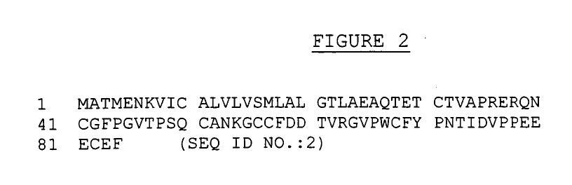

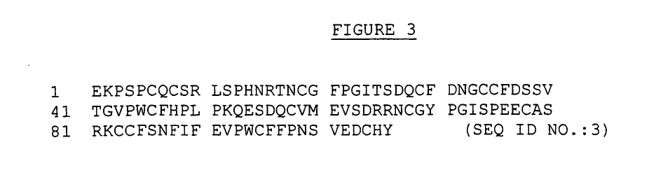

### DETAILED DESCRIPTION

The invention provides methods and compositions useful for the treatment, amelioration, and prevention of a wide range of lesions to the respiratory epithelium. Lesions of the respiratory epithelium treated according to the present invention can be caused by physical (e.g., surgical intervention or intubation), chemical (e.g., smoking or exposure to volatile solvent), or thermal trauma; vascular compromise (e.g., resulting from congestive heart failure or chronic obstructive pulmonary disease); infective or inflammatory processes; antineoplastic therapy (e.g., radiotherapy or chemotherapy); or other diseases processes such as cystic fibrosis or asthma, for example. Furthermore, another common chemical insult to the respiratory epithelium includes the exposure to high concentrations of oxygen (e.g., hyperbaric oxygen therapies) for extended periods of time.

Treatment of these lesions according to the invention can speed epithelial healing, reduce symptoms associated with the disruption to the airway epithelium, and reduce, delay or prevent the secondary complications of worsening rhinitis, asthma, pneumonitis, or other complications of the airway epithelial lesion. Further, since the invention will speed normal epithelial closure and reduce infection, it will reduce the chance of both acquiring secondary infections as well as late secondary effects of ongoing sensitization of the airway (e.g., hay fever and asthma).

Lesions of the respiratory epithelium, such as those resulting from allergic reactions or from physical trauma, are amenable to trefoil peptide therapy delivered as an aerosol or a dry powder. The composition is formulated (micronized) into a dry powder inhaler, or an aerosol according to known and conventional methods for preparing such formulations. When used to treat the tracheo-bronchial respiratory epithelium, administration of a composition of the invention preferably occurs as soon as symptoms occur and will last on the order of three to ten days, or alternatively until the lesion to the respiratory epithelium disappears. In the case of milder lesions however, trefoil peptide therapy may resolve the lesion in a shorter period of time, particularly when combined with another active ingredient.

The compositions of this invention can also be used prophylactically, prior to therapies that will damage the respiratory epithelium. For example, the compositions can be administered prior to anti-neoplastic therapy or prior to a surgical intervention in order to mitigate the loss of epithelial integrity. Prevention or amelioration of symptoms due to nasal-pharyngeal respiratory epithelial disruption may also be achieved by administering the trefoil peptide prior to the anticipated insult. For example, a patient may be administered trefoil peptide therapy before the exposure to tree or grass pollen in “hay fever” season, or by administering prophylactic treatment at reduced intervals, during the period when the patient is at risk for nasal-pharyngeal infections.

Typically, a metered dose inhaler or dry powder inhaler will be self-administered by the patient. Tidal breathing from a continuous nebulizer, usually under physician supervision, also allows for independent regulation of trefoil peptide and adjunct pharmaceutical dosages.

### Pharmaceutical Formulations

Aerosols

Aerosolized formulations deliver high concentrations of the trefoil peptide directly to the airways with low systemic absorption, and include for example nasal sprays, inhalation solutions, inhalation suspensions, and inhalation sprays. Nasal sprays typically contain a therapeutically active trefoil peptide dissolved or suspended in solution or in a mixture of excipients (e.g., preservatives, viscosity modifiers, emulsifiers, or buffering agents), in nonpressurized dispensers that deliver a metered dose of the spray. Inhalation solutions and suspensions are aqueous-based formulations containing the trefoil peptide and, if necessary, additional excipients. Such formulations are intended for delivery to the respiratory airways by inspiration. Typically, metered-dose aerosol inhalers create droplets that are 20 to 30 microns in diameter.

A major limitation of pulmonary delivery is the difficulty of reaching the deep lung. To achieve high concentrations of a trefoil peptide solution in both the upper and lower respiratory airways, the trefoil peptide is preferably nebulized in jet nebulizers, a ultrasonic nebulizer, or an electronic nebulizer particularly those modified with the addition of one-way flow valves, such as for example, the Pari LC Plus™ nebulizer, commercially available from Pari Respiratory Equipment, Inc., Richmond, Va., which delivers up to 20% more drug than other unmodified nebulizers.

The pH of the formulation is also important for aerosol delivery. When the aerosol is acidic or basic, it can cause bronchospasm and cough. The safe range of pH is relative and depends on a patient's tolerance. Some patients tolerate a mildly acidic aerosol, which in others will cause bronchospasm. Typically, an aerosol solution having a pH less than 4.5 induces bronchospasm. An aerosol solution having pH between 4.5 and 5.5 will occasionally cause this problem. The aerosol solution having a pH between 5.5 and 7.0 is usually considered safe. Any aerosol having pH greater than 7.0 is to be avoided as the body tissues are unable to buffer alkaline aerosols and result in irritation and bronchospasm. Therefore, the pH of the formulation is preferably maintained between 5.5 and 7.0, most preferably between 5.5 and 6.5 to permit generation of a trefoil peptide aerosol well tolerated by patients without any secondary undesirable side effects such as bronchospasm and cough. The osmolarity of the formulation can also be adjusted to osmolarities of about 250 to 350 mosm/L, according to the patient's tolerance. The administration of a hypertonic or a hypotonic solution may be poorly tolerated in certain instances, particularly when administered to a denuded mucosa. Propellants, such as HFA 134a, HFA 227, or combinations thereof, may also be used in the formulation. If desired, excipients that promote drug dispersion or enhance valve lubrication may also be formulated with the trefoil peptide.

Dry Powder Formulation

As an alternative therapy to aerosol delivery, the trefoil peptide may also be administered in a dry powder formulation for efficacious delivery into the endobronchial space. Such formulations have several advantages, including product and formulation stability, high drug volume delivery per puff, and low susceptibility to microbial growth. Therefore, dry powder inhalation and metered dose inhalation are most practical when high amounts of trefoil peptide need to be delivered, including for example cases in which a large portion of the respiratory epithelium is affected with lesions. Depending on the efficiency of the dry powder delivery device, effective dry powder dosage levels typically fall in the range of about 20 to about 60 mg. The invention therefore provides a sufficiently potent formulation of a trefoil peptide in dry powder or metered dose form of drug particles. Such a formulation is convenient because it does not require any further handling such as diluting the dry powder. Furthermore, it utilizes devices that are sufficiently small, fully portable and tend to have a long shelf life.

For dry powder formulations of the invention, a trefoil peptide composition is milled to a powder having mass median aerodynamic diameters ranging from 1-10 microns by media milling, jet milling, spray drying, super-critical fluid energy, or particle precipitation techniques.

Particle size determinations may be made using a multi-stage Anderson cascade impactor or other suitable method. Alternatively, the dry powder formulation may be prepared by spray drying or solution precipitation techniques. Spray drying has the advantage of being the least prone to degrading the trefoil peptides. Solution precipitation is performed by adding a co-solvent that decreases the solubility of a drug to a uniform drug solution. When sufficient co-solvent is added the solubility of the drug falls to the point where solid drug particles are formed which can be collected by filtration or centrifugation. Precipitation has the advantage of being highly reproducible and can be performed under low temperature conditions, which reduce degradation. Super-critical fluid technology can produce particles of pharmaceutical compounds with the controlled size, density and crystallinity ideal for powder formulations.

The dry powder formulations of the present invention may be used directly in metered dose or dry powder inhalers. Currently, metered dose inhaler technology is optimized to deliver masses of 1 microgram to 5 mg of a therapeutic. Spacer technology, such as the aerochamber, may also be utilized to enhance pulmonary exposure and to assist patient coordination.

An alternate route of dry powder delivery is by dry powder inhalers. There are two major designs of dry powder inhalers, device-metering designs in which a reservoir of drug is stored within the device and the patient ‘loads’ a dose of the device into the inhalation chamber, and the inspiratory flow of the patient accelerates the powder out of the device and into the oral cavity. Alternatively, dry powder inhalers may also employ an air source, a gas source, or electrostatics, in order to deliver the trefoil peptide. Current technology for dry powder inhalers is such that payload limits are around 10 mg of powder. The dry powder formulations are temperature stable and have a physiologically acceptable pH of 4.0-7.5, preferably 6.5 to 7.0.

### Therapeutic Agents

In addition to the trefoil peptide, the therapeutic formulation according to the present invention may also comprise a second therapeutic agent, or regimen. The second therapeutic agent may be administered within (either before, or after administration of the trefoil peptide) 14 days, 7 days, 1 day, 12 hours, 1 hour, or simultaneously with the trefoil peptide. The second therapeutic agent can also be present in the same or different pharmaceutical compositions as the trefoil peptide. Thus, pharmaceutical compositions for locally treating the respiratory epithelium may include, in addition to a trefoil peptide, for example, an anti-inflammatory compound, an antibiotic, a beta-adrenergic bronchodilator, a cholinergic receptor antagonist, a neurokinin receptor antagonist, a steroid, a decongestant, a phosphodiesterase inhibitor, an analgesic, or an anesthetic. When the second therapeutic agent is present in a different pharmaceutical composition, different routes of administration may be used. For example, the second therapeutic agent may be administered orally, or by intravenous, intramuscular, or subcutaneous injection. Thus, the second therapeutic agent need not be administered by inhalation. If desired, more than one therapeutic agent may be administered with the trefoil peptide. Of course, pharmaceutical compositions may also contain two, three, or more trefoil peptides, or biologically active fragments.

### Trefoil Peptides

The therapeutic trefoil peptide(s) are typically mammalian trefoil peptides or fragments thereof although non-naturally occurring homologs that are substantially identical to the mammalian trefoil peptides are also useful. Preferably, human trefoil peptides or fragments are used; however, trefoil peptides from other species including rat, mouse, and non-human primate, may be used. Typically, the trefoil peptide is intestinal trefoil factor (ITF); however, spasmolytic polypeptide (SP), or pS2 are also useful.

Particular trefoil peptide fragments retain biological activity and may be substituted in any method or composition in which a trefoil peptide is used. Methods and compositions containing a trefoil peptide, in which these trefoil peptide fragments may be substituted, are described, for example, in U.S. Pat. Nos. 6,063,755 and 6,221,840, and U.S. patent application Ser. Nos. 10/131,363, filed Apr. 24, 2002, 60/317,657, filed Sep. 6, 2001, 60/327,673, filed Oct. 5, 2002, 60/333,836, filed Nov. 28, 2001, and 60/367,574, filed Mar. 26, 2002 (hereby incorporated by reference).

Particularly useful ITF fragments that retain biological activity include the polypeptide corresponding to amino acid residues 15-73 of SEQ ID NO: 1 (ITF₁₅₋₇₃) and amino acid residues 21-73 of SEQ ID NO:1 (ITF₂₁₋₇₃). Other useful ITF fragments are formed following cleavage of the C-terminal phenylalanine residue (i.e., ITF₁₋₇₂, ITF₁₅₋₇₂, and ITF₂₁₋₇₂).

The biologically active trefoil peptide fragments of this invention can be produced using any appropriate method. For example, cDNA encoding the desired ITF fragment can be used with any method known in the art for producing recombinant proteins. Exemplary methods are provided herein. ITF fragments, particularly ITF₂₁₋₇₃, can be produced using a Pichia yeast expression system (see, for example, U.S. Pat. Nos. 4,882,279 and 5,122,465) transformed with a cDNA encoding long ITF species, such as the full length ITF (e.g., SEQ ID NO: 4) or ITF₁₅₋₇₃, when the fermentation culture is maintained at pH˜5.0.

The trefoil peptides, including ITF, are soluble, and can therefore be dissolved in a pharmaceutically acceptable carrier liquid for aerosolization or nebulization for example. Aerosols containing a trefoil peptide are optimized for aerodynamic particle size, to target airway regions of interest. Typically aerosol sizes of 1-3 micron target deep lung (alveolar) structures, while a particle size of 5-10 micron result in tracheo-bronchial deposition. Moreover certain excipients may be used to prolong the local release of a trefoil peptide delivered in the lung or nasal region, or to retain the trefoil peptide formulation in the desired local area of the lung by modifying the mucociliary clearance rate.

### Trefoil Peptide Dosages

Typically, the dosage, frequency and duration of therapy are tailored to the type and severity of the lesion being treated. For example, intermittent dosing may be sufficient to treat minor airway lesions. More severe airway lesions, resulting from, for example, severe smoke inhalation or thermal damage, may require continuous trefoil peptide administration. Alternatively, treatment may also be administered prophylactically, in anticipation of lesions to the respiratory epithelium. The prophylactic treatment may begin at least one day, three days, five days, seven days, or ten days prior to the insult. Treatment of unanticipated airway lesions preferably begin immediately after insult, or within 24 hours. Preferably, trefoil peptide therapy is administered at least one, two, three, four, or more than four times per day for at least one day, five days, fourteen days, or even for the lifetime of the patient being treated. Alternatively, the trefoil peptide may be continuously administered to the patient over a set period of time, for a duration of one hour, two hours, 6 hours, one day, or more than one day for example. For this purpose, the trefoil peptide may be administered using a mask adapter of a nebulizer system, for example.

Preferably, aerosol formulation contains a trefoil peptide concentration of 5, 10, 20, 40, 60, 80, 100 mg/mL, or more and is formulated in a physiologically acceptable solution, preferably in one quarter strength of normal saline. Ideally, the patient is administered with at least 10, 50, 100, 200, 500, 700, 1000, or more than 1000 micrograms of a trefoil peptide administered as an aerosol. The use of dry powder inhalation preferably results in the delivery of at least about 1, 5, 10, 20, 30, 40, 50, 60, or more than 60 mg of the trefoil peptide to the respiratory airways of the patient receiving treatment. In such a formulation, the trefoil peptide is delivered as a powder in an amorphous or crystalline state in particle sizes between 1 and 10 microns in mass median aerodynamic diameter necessary for efficacious delivery of the trefoil peptide into the endobronchial space for treatment, amelioration, and prevention of lesions of the respiratory epithelium. Fractions of 2 to 4 microns may also be employed to target the peripheral lung. Patient inspiration techniques, such as breath holding for example, may also optimize deposition of the trefoil peptide.

If desired, the trefoil peptide may also be administered orally, or by intravenous injection, particularly in cases in which controlled or continuous release of the trefoil peptide is the goal.

All of the therapeutic agents employed in the compositions of the present invention, including the trefoil peptide component, can be used in the dose ranges currently known and used for these agents. Different concentrations of either the trefoil peptide or the other agents may be employed depending on the clinical condition of the patient, the goal of the therapy (treatment or prophylaxis), the anticipated duration, the lesion site, and the severity of the damage for which the trefoil peptide is being administered. Additional considerations in dose selection include: disease etiology, patient age (pediatric, adult, geriatric), general health and comorbidity.

### Anti-Inflammatory Agents

Any suitable anti-inflammatory agent can be formulated with the trefoil peptide and employed using the method of this invention. Suitable anti-inflammatory agents can be administered systemically, or can be administered by inhalation. Exemplary agents include, but are not limited to non-steroidal anti-inflammatory drugs (e.g., ibuprofen, tacrolimus, Cromolyn, Nedocromil), cyclooxygenase-2-specific inhibitors such as rofecoxib (Vioxx®) and celecoxib (Celebrex®), and glucocorticoids. Particularly effective glucocorticosteroid agents that may be used by aerosolization include for example beclomethasone, flunisolide, budesonide and triamcinolone. Other useful glucocorticoisteroid agents include prednisolone, dexamethasone and fluticasone. Although asthma is the main lung condition in which corticosteroids are used, such agents may also be useful when the respiratory epithelium is damaged by cigarette smoke as in chronic bronchitis and emphysema for example. Corticosteroids are also useful in the treatment of other lung diseases such as sarcoidosis, alveolitis and chronic inflammatory conditions. These drugs may be given orally, intravenously (e.g., in severe cases), or by inhalation. Preferably, inhaled corticosteroids are administered to the patient because the dose required is much less and is delivered directly to the small air passages in the lungs with fewer associated side effects.

Anti-inflammatory concentrations known to be effective following inhalation administration can be used. For example, ibuprofen may be present in the composition at concentrations sufficient to deliver between 25-800 mg per day to the respiratory lesion.

### Bronchodilator Agents

Any active bronchodilator agent may be co-formulated with the trefoil peptide in the usual doses for respiratory application to the nasal-pharyngeal or tracheo-bronchial anatomy. Useful bronchodilators include, but are not limited to methylxanthines (e.g., theophylline, theobromine, and caffeine), sympathomimetic agents (e.g., adrenaline, epinephrine, isoproterenol, and beta-adrenergic agonists), cholinergic receptor antagonists such as ipratroprium bromide and tiotropium and neurokinin receptor antagonists.

Adrenergic bronchodilators are usually administered by inhalation to open up the bronchial tubes (air passages) of the lungs and are typically used to treat, ameliorate, or prevent the symptoms of asthma, chronic bronchitis, emphysema, and other lung diseases. Such exemplary bronchodilators include albuterol, bitolterol, epinephrine, fenoterol, formoterol, isoetharine, isoproterenol, metaproterenol, pirbuterol, procaterol, racepinephrine, salmeterol, and terbutaline.

Alternatively, the trefoil peptide of the invention may be administered with a leukotriene receptor antagonist (e.g., montelukast, or zafirlukast), a neurokinin receptor antagonist, an antihistamine (e.g., diphenhydramine, fexofenadine, cetirizine, or loratadine) or a cholinergic receptor antagonist.

### Antimicrobial Agents

Any suitable antimicrobial agent can be used in the compositions of the invention at concentrations generally used for these agents. Suitable antimicrobial agents include, antibacterial, antifungal, antiparasitic, and antiviral agents. Exemplary antibacterial agents (antibiotics) include the penicillins (e.g., penicillin G, ampicillin, methicillin, oxacillin, and amoxicillin), the cephalosporins (e.g., cefadroxil, ceforanid, cefotaxime, and ceftriaxone), the tetracyclines (e.g., doxycycline, minocycline, and tetracycline), the aminoglycosides (e.g., amikacin, gentamycin, kanamycin, neomycin, streptomycin, and tobramycin), the macrolides (e.g., azithromycin, clarithromycin, and erythromycin), the fluoroquinolones (e.g., ciprofloxacin, lomefloxacin, and norfloxacin), and other antibiotics including chloramphenicol, clindamycin, cycloserine, isoniazid, rifampin, and vancomycin. Particularly useful formulations contain aminoglycosides, including for example amikacin, gentamicin, kanamycin, neomycin, netilmicin, paromomycin, streptomycin, and tobramycin.

Antiviral agents are substances capable of destroying or suppressing the replication of viruses. Examples of anti-viral agents include 1-D-ribofuranosyl-1,2,4-triazole-3 carboxamide, 9->2-hydroxy-ethoxy methylguanine, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogues such as acyclovir, penciclovir, valacyclovir, and ganciclovir.

Antifungal agents include both fungicidal and fungistatic agents such as, for example, benzoic acid, undecylenic alkanolamide, ciclopirox olamine, polyenes, imidazoles, allylamine, thicarbamates, amphotericin B, butylparaben, clindamycin, econaxole, fluconazole, flucytosine, griseofulvin, nystatin, and ketoconazole.

Other antimicrobial agents such as the antiparasitics like pentamidine, are known to have respiratory side effects. Therefore, co-administration of a trefoil peptide and an antimicrobial of this type may reduce or prevent adverse events.

Antimicrobial concentrations known to be effective in treating respiratory infections can be used.

### Anticancer Agents

Cancers of the lung, including small cell and non-small cell carcinomas, damage the lung epithelium. Frequently, this injury is exacerbated by anticancer therapy because many anticancer agents have adverse effects on epithelial cells. Therefore, it is beneficial to administer trefoil peptide therapy in anticipation of, concurrent to, or following antineoplastic therapy to prevent, ameliorate, or treat damage to the respiratory epithelium. Chemotherapeutics are usually administered systemically by intravenous injection. The trefoil peptides may administered simultaneously, as an additive to the chemotherapeutic preparation, or separately, by inhalation. For patients undergoing radiation therapy, trefoil peptides are preferably administered by inhalation beginning one to three days prior to each therapeutic session, continuing through the course of therapy, and continuing for one to three days after the final radiation treatment.

### Production of Trefoil Peptides

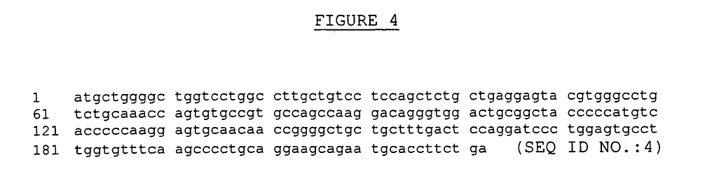

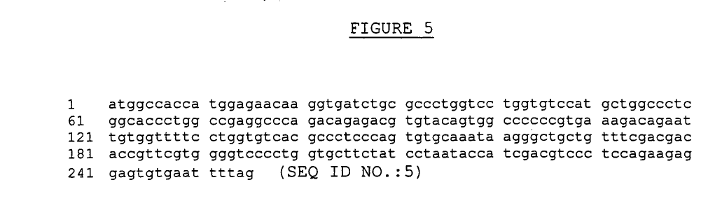

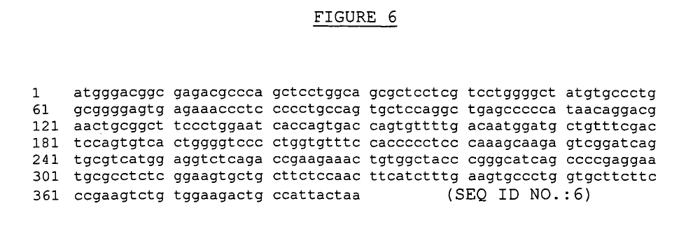

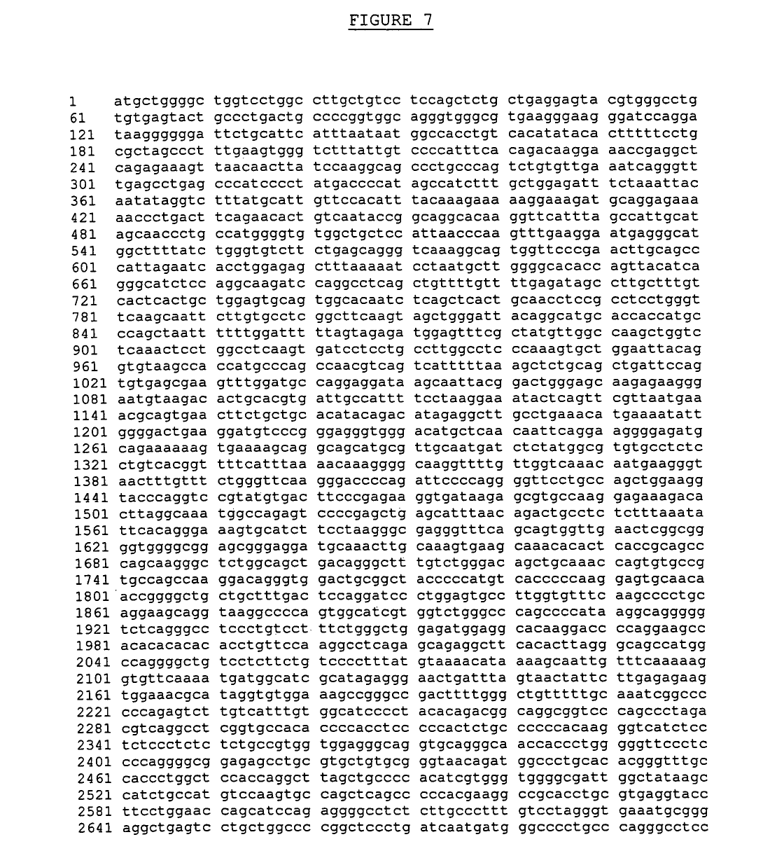

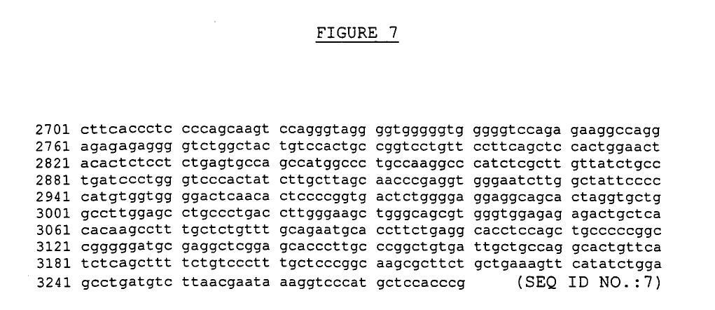

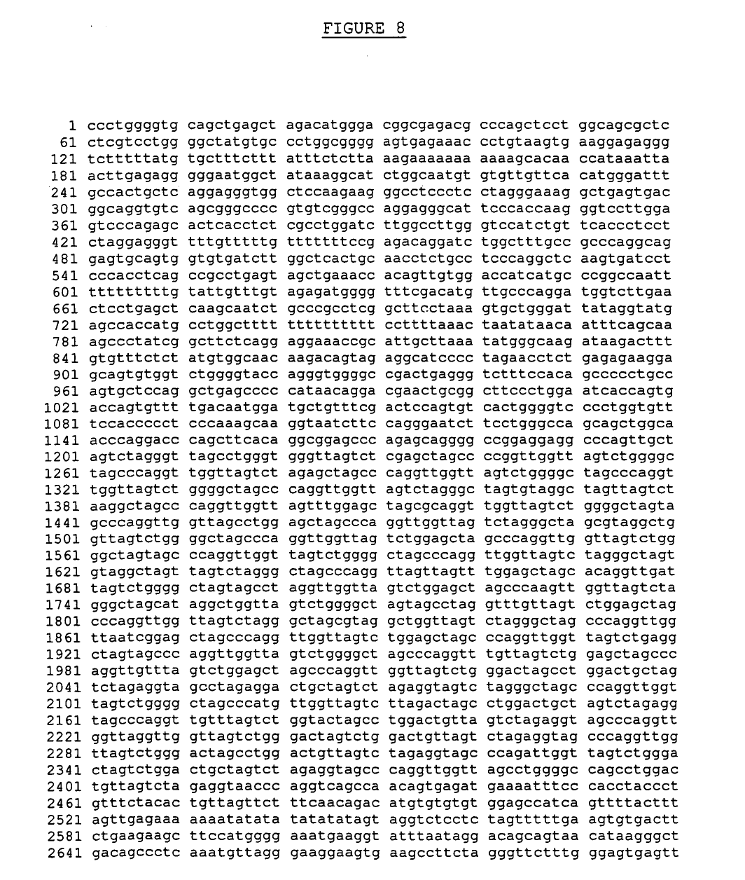

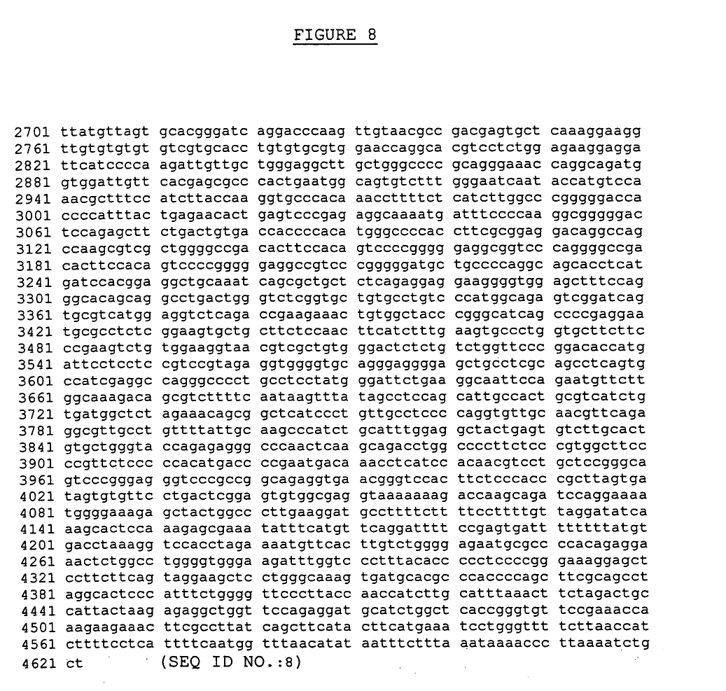

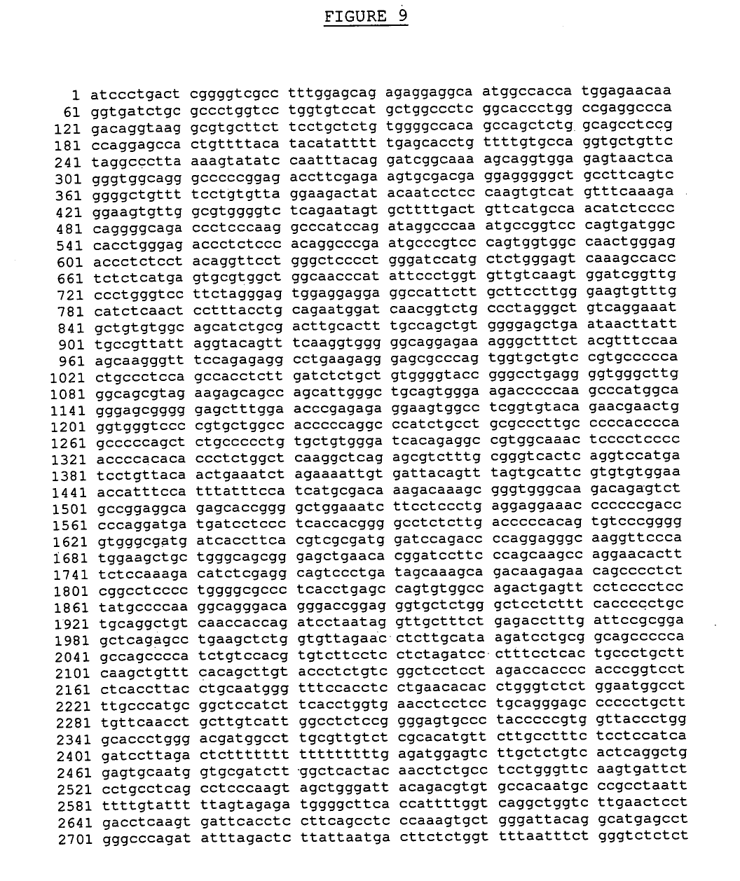

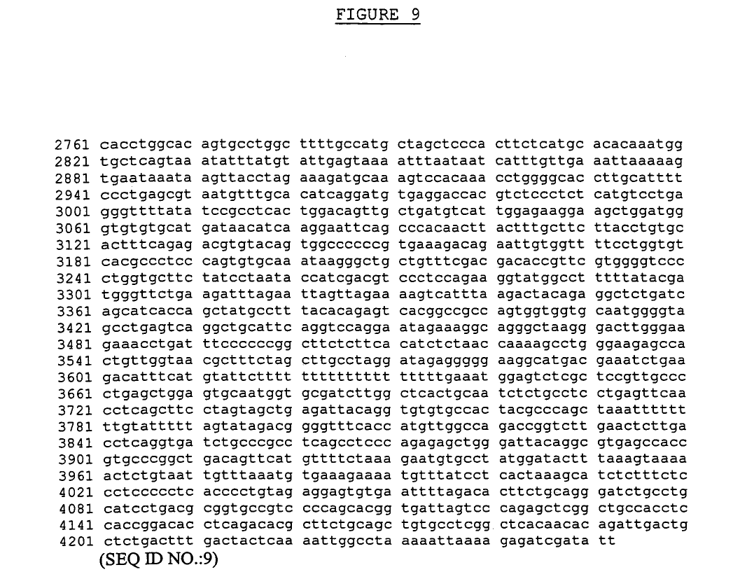

Trefoil peptides and fragments can be produced by any method known in the art for expression of recombinant proteins. Nucleic acids that encode trefoil peptides (e.g., human intestinal trefoil factor (FIGS. 4 and 7), human pS2 (FIGS. 5 and 8), and human spasmolytic polypeptide (FIGS. 6 and 9) or fragments thereof may be introduced into various cell types or cell-free systems for expression thereby allowing large-scale production, purification, and patient therapy.

Eukaryotic and prokaryotic trefoil peptide expression systems may be generated in which a trefoil peptide gene sequence is introduced into a plasmid or other vector, which is then used to transform living cells. Constructs in which the trefoil peptide cDNA contains the entire open reading frame inserted in the correct orientation into an expression plasmid may be used for protein expression. Prokaryotic and eukaryotic expression systems allow for the expression and recovery of trefoil peptide fusion proteins in which the trefoil peptide is covalently linked to a tag molecule, which facilitates identification and/or purification. An enzymatic or chemical cleavage site can be engineered between the trefoil peptide and the tag molecule so that the tag can be removed following purification.

Typical expression vectors contain promoters that direct the synthesis of large amounts of mRNA corresponding to the inserted trefoil peptide nucleic acid in the plasmid-bearing cells. They may also include a eukaryotic or prokaryotic origin of replication sequence allowing for their autonomous replication within the host organism, sequences that encode genetic traits that allow vector-containing cells to be selected for in the presence of otherwise toxic drugs, and sequences that increase the efficiency with which the synthesized mRNA is translated. Stable long-term vectors may be maintained as freely replicating entities by using regulatory elements of, for example, viruses (e.g., the OriP sequences from the Epstein Barr Virus genome). Cell lines may also be produced that have integrated the vector into the genomic DNA, and in this manner the gene product is produced on a continuous basis.

Expression of foreign sequences in bacteria, such as Escherichia coli, requires the insertion of a trefoil peptide nucleic acid sequence into a bacterial expression vector. Such plasmid vectors contain several elements required for the propagation of the plasmid in bacteria, and for expression of the DNA inserted into the plasmid. Propagation of only plasmid-bearing bacteria is achieved by introducing, into the plasmid, selectable marker-encoding sequences that allow plasmid-bearing bacteria to grow in the presence of otherwise toxic drugs. The plasmid also contains a transcriptional promoter capable of producing large amounts of mRNA from the cloned gene. Such promoters may be (but are not necessarily) inducible promoters that initiate transcription upon induction. The plasmid also preferably contains a polylinker to simplify insertion of the gene in the correct orientation within the vector.

Mammalian cells can also be used to express a trefoil peptide. Stable or transient cell line clones can be made using trefoil peptide expression vectors to produce the trefoil peptides in a soluble (truncated and tagged) form. Appropriate cell lines include, for example, COS, HEK293T, CHO, or NIH cell lines.

Once the appropriate expression vectors are constructed, they are introduced into an appropriate host cell by transformation techniques, such as, but not limited to, calcium phosphate transfection, DEAE-dextran transfection, electroporation, microinjection, protoplast fusion, or liposome-mediated transfection. The host cells that are transfected with the vectors of this invention may include (but are not limited to) E. coli or other bacteria, yeast, fungi, insect cells (using, for example, baculoviral vectors for expression in SF9 insect cells), or cells derived from mice, humans, or other animals. In vitro expression of trefoil peptides, fusions, or polypeptide fragments encoded by cloned DNA may also be used. Those skilled in the art of molecular biology will understand that a wide variety of expression systems and purification systems may be used to produce recombinant trefoil peptides and fragments thereof. Some of these systems are described, for example, in Ausubel et al. (Current Protocols in Molecular Biology, John Wiley & Sons, New York, N.Y. 2000, hereby incorporated by reference).

Transgenic plants, plant cells and algae are also particularly useful for generating recombinant trefoil peptides for use in the methods and compositions of the invention. For example, transgenic tobacco plants or cultured transgenic tobacco plant cells expressing a trefoil peptide can be created using techniques known in the art (see, for example, U.S. Pat. Nos. 5,202,422 and 6,140,075). Transgenic algae expression systems can also be used to produce recombinant trefoil peptides (see, for example, Chen et al., Curr. Genet. 39:365-370, 2001).

Once a recombinant protein is expressed, it can be isolated from cell lysates using protein purification techniques such as affinity chromatography. Once isolated, the recombinant protein can, if desired, be purified further by e.g., high performance liquid chromatography (HPLC; e.g., see Fisher, Laboratory Techniques In Biochemistry And Molecular Biology, Work and Burdon, Eds., Elsevier, 1980).

Polypeptides of the invention, particularly trefoil peptide fragments can also be produced by chemical synthesis using, for example, Merrifield solid phase synthesis, solution phase synthesis, or a combination of both (see, for example, the methods described in Solid Phase Peptide Synthesis, 2nd ed., 1984, The Pierce Chemical Co., Rockford, Ill.). Optionally, peptide fragments are then be condensed by standard peptide assembly chemistry.

The following examples are intended to illustrate the principle of the present invention and circumstances when trefoil peptide therapy is indicated. The following examples are not intended to be limiting.

### EXAMPLE 1

### Treatment of Rhinitis Due to Rhinovirus

The patient is administered a trefoil peptide-containing preparation beginning immediately after the onset of a head cold. The preparation contains a therapeutic dose of ITF₁₅₋₇₃. The trefoil peptide can be administered as a nasal spray using standard formulating methods to deliver 100 microliters of a 50 mg/ml spray of trefoil peptide. The patient receives medication by self-administering the nasal spray every 12 hours for the next five consecutive days. Also, the trefoil peptide active material may be applied with the standard dose of a nasal decongestant spray (e.g. 0.05% oxymetazoline HCl).

### EXAMPLE 2

### Treatment of Allergic Rhinitis Due to Grass Pollen

During hay fever season, the patient affected with allergic rhinitis is administered with antihistamines such as diphenhydramine, fexofenadine, cetirizine, or loratadine. Also, the patient is concurrently administered a nasal spray preparation containing a therapeutic dose of ITF₁₅₋₇₃. This component, in one example, is a nasal spray using standard formulating methods to deliver a 5 mg/ml spray of ITF. Continuing for the subsequent five days, the patient receives medication by self-administered nasal spray every 12 hours or as needed. In severe cases, the ITF active material may further be applied with the standard dose of a nasal glucocorticoid spray (e.g., beclomethasone, fluticasone, mometasone, or triamcinolone).

### EXAMPLE 3

### Treatment of a Post Viral Prolonged Bronchospasm

In treatments for post-viral tracheo-bronchial epithelial disruption, the trefoil peptide containing material may be co-formulated with the standard dose of an inhaled salmeterol preparation, in a dry powder inhaler, an aerosol metered dose inhaler, or as a solution or a suspension in a ultrasonic or air-jet nebuliser. The treatment continues with the patient self-administering the medication every 12 hours for a period of at least 72 hours.

### EXAMPLE 4

### Treatment of Adult Respiratory Distress Syndrome (ARDS)

Acute respiratory distress syndrome (ARDS) is a characteristic response of the lung in reaction to a wide variety of injury. Treatment of ARDS is initiated as soon as possible to minimize damage caused to the lung. The objective of treatment is to provide enough support for the failing respiratory system (and other systems) until these systems have time to heal. The main supportive treatment of the failing respiratory system in ARDS is mechanical ventilation (a breathing machine) to deliver high doses of oxygen and a continuous level of pressure called PEEP (positive end-expiratory pressure) to the damaged lungs. To speed healing, a trefoil peptide is administered by inhalation to patients with established ARDS or a syndrome of pre-ARDS. The amount of ITF₂₁₋₇₃ will be on the order of 1000 mg every 24 hours. The treatment is continued for at least 72 hours depending on the severity of the case and the clinical response of the patient. The regimen is repeated until healing or for ten days of therapy. It may be more convenient to administer trefoil proteins to these patients less frequently (e.g. every 12 or 24 hours) and in higher concentrations with or without formulations to enhance the exposure of the lung capillary epithelium to the peptide. Additional forms of treatment that may be used along with the trefoil peptide therapy include for example antibiotics, immunosuppressants, blood pressure supporting medications, tube feedings, and diuretics, which are used to reduce the fluid in the lungs. Since the pathology of ARDS is also linked to excessively produced nitric oxide, a NO blocker may be administered, if desired.

### EXAMPLE 5

### Treatment of Human Respiratory Syncitial Virus

Human respiratory syncitial virus is the most important cause of hospitalizations for viral respiratory tract disease in young children worldwide. Primary infection usually causes upper respiratory symptoms. Although the infection initiates in the upper respiratory tract, it can spread to the lower tract, via aspiration of secretions or via the respiratory epithelium, causing bronchiolitis and pneumonia. During the infection, RSV causes extensive damage to the epithelium and the bronchiolar ciliary apparatus. Children affected by RSV may be administered ITF therapy to accelerate recovery of the respiratory epithelium. Patients are administered a trefoil peptide by inhalation, using for example, a dry powder inhaler, an aerosol metered dose inhaled, a solution or a suspension in a ultrasonic or air-jet nebuliser. The trefoil peptide is administered three times a day, at a dose of 1 mg/puff. Desirably, Ribavirin, an aerosolized drug that can reduce the severity and the duration of illness, is also administered.

### EXAMPLE 6

### Treatment of Influenza Infection

The influenza virus infects epithelial cells of the trachea and the bronchi. Extensive damage to the epithelium due to infection can cause severe coughing as well as pain in the chest, and the release of cytokines from damaged cells can further cause fever, chills, malaise, and muscular pains. Also, severe destruction of the mucous epithelium may lead to secondary bacterial infection and bronchitis. To alleviate the symptoms and accelerate the rate of recovery, the patient is administered trefoil peptide therapy as soon as symptoms of infection are manifested. ITF, or a biologically active fragment thereof, is administered in a dry powder inhaler, an aerosol metered dose inhaled, or as a solution or a suspension in an ultrasonic or air-jet nebuliser. Alternatively, patients may also be administered the trefoil peptide therapy by a nasal spray. This therapy is administered three to four times a day, and may be continued for a week following dissipation of the symptoms.

### EXAMPLE 7

### Treatment of Chronic Bronchitis

Chronic Bronchitis is typically caused by chronic irritation of the respiratory airways or by microbial infections. As such, it is a condition often associated with smoking and its incidence is often associated with emphysema. Patients typically have a chronic cough with sputum. Damage to the epithelium from chronic bronchitis may predispose individuals to pneumococcal bacterial invasion, which can lead to further complications, such as pneumonia. Therefore, restoration or improvement of the respiratory epithelium can alleviate symptoms associated with chronic bronchitis. Patients diagnosed with chronic bronchitis, or smokers, are immediately administered with a trefoil peptide in a dry powder inhaler, an aerosol metered dose inhaled, or as a solution or a suspension in an ultrasonic or air-jet nebuliser. Patients can self-administer this regimen at least three times a day, for a period of at least seven days, or until the coughing ceases. If desired, the trefoil peptide therapy may also include administration of antibiotics.

### EXAMPLE 8

### Treatment of Lesions Caused by Smoke Inhalation

Direct toxic effects caused by rapidly acting toxins such as smoke can incapacitate patients within moments. As such, the resulting effects, which include bronchospasm and alveolar damage, may cause rapid deterioration of the patient and high mortalities. Inhalation of smoke can initiate an inflammatory response in a patient causing the release of histamine and other vasoactive substances that cause damage to the respiratory epithelium. Treatment will vary with the severity of the damage caused by smoke inhalation. The primary focus of treatment is to maintain an open airway and provide an adequate level of oxygen. If the airway is open and stable, the patient may be given high-flow humidified 100% oxygen by mask. If swelling of the airway tissues is closing off the airway, the patient may require the insertion of an endotracheal tube to artificially maintain an open airway.

The patient is also immediately and continuously administered ITF₁₅₋₇₃ by jet nebulizer for at least five days to reduce smoke-induced damage to the airway epithelium and the deleterious effects of hyperbaric oxygen therapies.

### EXAMPLE 9

### Treatment of Asthma

The management of asthma is concerned primarily with the relief and prevention of symptoms through the treatment of underlying inflammatory processes, which cause damage to the respiratory epithelium. Furthermore, if untreated, chronic inflammation makes the airways hyper-responsive to stimuli such as cold air, exercise, dust mites, pollutants in the air, thus exacerbating damage to the epithelium. Consequently, the asthmatic patient is administered with theophylline, an anti-inflammatory agent and a therapeutically effective amount of ITF₁₅₋₇₃ to ameliorate asthma-associated symptoms and to reduce damage to the respiratory airways.

What is claimed is:

1. A method for treating asthma in a mammal, comprising administering to said mammal a composition comprising a therapeutically effective amount of a trefoil peptide, or a biologically active fragment thereof.

2. The method of claim 1, wherein said trefoil peptide is intestinal trefoil factor (ITF), spasmolytic polypeptide, or pS2.

3. The method of claim 1, wherein said biologically active fragment is a fragment of intestinal trefoil factor (ITF) and is selected from the group consisting of ITF₁₅₋₇₃, ITF₂₁₋₇₃, ITF₁₋₇₂, ITF₁₅₋₇₂, and ITF₂₁₋₇₂, wherein the bounds of said fragment are delineated according to SEQ ID NO.: 1.

4. The method of claim 3, wherein said biologically active fragment is ITF₁₅₋₇₃.

5. The method of claim 1, wherein said administration is by inhalation.

6. The method of claim 1, wherein said composition further comprises a second therapeutic agent.

7. The method of claim 6, wherein said second therapeutic agent is an anti-inflammatory agent, antimicrobial agent, antihistamine, neurokinin receptor antagonist, leukotriene receptor antagonist, decongestant, cholinergic receptor antagonist, phosphodiesterase inhibitor, or a beta-adrenergic bronchodilator.

8. The method of claim 7, wherein said anti-inflammatory agent is a glucocorticoid.

9. The method of claim 6, wherein said beta-adrenergic bronchodilator is albuterol, bitolterol, epinephrine, fenoterol, formoterol, isoetharine, isoproterenol, metaproterenol, pirbuterol, procaterol, racepinephrine, salmeterol, or terbutaline.

10. The method of claim 1, wherein said biologically active fragment of a trefoil peptide is encoded by an isolated nucleic acid molecule that hybridizes under high stringency conditions to a polynucleotide having the sequence of SEQ ID NO.: 4, SEQ ID NO.: 5, SEQ ID NO.: 6, SEQ ID NO.: 7, SEQ ID NO.: 8, or SEQ ID NO.: 9.

11. A pharmaceutical composition suitable for inhalation administration for the treatment of asthma in a mammal, wherein said composition comprises a trefoil peptide, or a biologically active fragment thereof, and a pharmaceutically acceptable carrier.

12. The composition of claim 11, wherein said trefoil peptide is intestinal trefoil factor (ITF), spasmolytic polypeptide, or pS2.

13. The composition of claim 11, wherein said biologically active fragment is ITF₁₅₋₇₃, ITF₂₁₋₇₃, ITF₁₋₇₂, ITF₁₅₋₇₂, or ITF₂₁₋₇₂.

14. The composition of claim 11, wherein said biologically active fragment is ITF₁₅₋₇₃.

15. The composition of claim 11, wherein said composition is an aerosol or a dry powder.

16. The composition of claim 11, wherein said composition further comprises a second therapeutic agent.

17. The composition of claim 16, wherein said second therapeutic agent is an anti-inflammatory agent, antimicrobial agent, antihistamine, neurokinin receptor antagonist, leukotriene receptor antagonist, decongestant, cholinergic receptor antagonist, phosphodiesterase inhibitor, or a beta-adrenergic bronchodilator.

18. The composition of claim 17, wherein said anti-inflammatory agent is a glucocorticoid.

19. The composition of claim 17, wherein said beta-adrenergic bronchodilator is albuterol, bitolterol, epinephrine, fenoterol, formoterol, isoetharine, isoproterenol, metaproterenol, pirbuterol, procaterol, racepinephrine, salmeterol, or terbutaline.

20. The composition of claim 11, wherein said trefoil peptide is encoded by an isolated nucleic acid sequence that hybridizes under high stringency conditions with the sequence of SEQ ID NO.: 4, SEQ ID NO.: 5, SEQ ID NO.: 6, SEQ ID NO.: 7, SEQ ID NO.: 8, or SEQ ID NO.: 9.
